(19) United States
(12) Patent Application Publication
(10) Pub. No.: US 2016/0193068 A1
(43) Pub. Date: Jul. 7, 2016

(54) DOUBLE SOFT COLLAR

(71) Applicant: Kumara Venkatanarayana Nibhanipudi, Hyderabad (IN)

(72) Inventor: Kumara Venkatanarayana Nibhanipudi, Hyderabad (IN)

(21) Appl. No.: 14/980,595

(22) Filed: Dec. 28, 2015

(30) Foreign Application Priority Data
Jan. 2, 2015 (IN) 34/CHE/2015

(51) Int. Cl. A61F 5/055

(52) U.S. Cl. CPC A61F 5/055

(57) ABSTRACT

The present invention relates to medical devices in the field of Emergency and Intensive Care Medicine. The present invention particularly discloses the method of use of the double soft cervical collar. The present invention also particularly relates to the usefulness of double soft cervical collar over hard (Philadelphia collar) and soft cervical collars.

### FIELD OF INVENTION

The present invention relates to medical devices in the field of Emergency and Intensive Care Medicine. The present invention particularly discloses the method of use of the double soft cervical collar. The present invention also particularly relates to the usefulness of double soft cervical collar over hard (Philadelphia collar) and soft cervical collars.

### BACKGROUND OF INVENTION

Neck consists of bones, muscles and ligaments. Any abnormality, inflammation, strain, injury, or trauma can cause neck pain. Neck pain is seldom a debilitating condition, though it can indicate serious underlying physiological or pathological conditions when it extends beyond a week.

In either case, cervical collars are the one of the basic prescriptions besides analgesics. Cervical collars, which are orthopaedic medical devices, are preferred for the mere reason of physical support and to facilitate immobility of the neck that will reduce further trauma, and realign the spinal cord and thus relieve pain. However, these are not recommended to be worn for long periods of time.

Cervical collars are of two main types—hard (Philadelphia collar) and soft.

The hard cervical collars, on one hand, are rigid, wrapped tightly around the neck. They find specific use in Emergency Medicine after severe trauma, surgery, cervical fractures, or dislocations because they are highly restrictive of the neck movements. On the other hand, they can cause adverse affects like exacerbation of pain, breath restrictions, tissue ischemia, increased risk of aspiration, on continuous use for a long time and can also present difficulty in nursing care and discomfort to the patient.

Soft cervical collars are a contrast to the hard cervical collars. They are flexible with contours that facilitate easy alignment around the neck. They are soft when wrapped around the neck and do not give a tightened feeling and hence, are the most comfortable collars for a patient. They are, however, least restrictive of the neck movements and the scope of use is only in the less serious conditions like strain, sprain, whiplash injury, or chronic neck pain that can be caused by different reasons. They are cheaper compared to the hard cervical collars.

In an article published in Curr Rev Musculoskelet Med. in June 2008; 1(2): 114-119 published online on Dec. 12, 2007. doi: 10.1007/s12178-007-9017-9, titled ‘When should a cervical collar be used to treat neck pain?’, authored by Stefan Muzin et al., it was disclosed that while hard collars were an important part of acute injury and spine stabilization, they were not routinely used to manage pain because of severe adverse effects on prolonged use.

In an article published in the J Orthop Sports Phys Ther. in 1996 March; 23(3):209-15, titled ‘The effect of a soft collar, used as normally recommended or reversed, on three planes of cervical range of motion’, authored by Carter V M, et al., it was stated that the soft collars, recommended to immobilize the cervical spine following trauma, could be used in normal or reversed manner, the latter method being useful to achieve limitation of mobility in a specific direction, without drastic differences in the extent of neck movement immobilization. The study was stated to be done on 50 healthy volunteers for investigating the effectiveness of soft cervical collars in limiting cervical range of motion under three conditions which being no collar, recommended use, and reversed manner, using the Orthopaedic Systems Inc. Computerized Anatometry-6000 Spine Motion Analyzer in six ranges of motions. It was disclosed that the post hoc paired t tests showed that wearing the collar either as recommended or reversed manner, decreased motion compared with not wearing a collar, and that the position of the collar affected range of motion in three of the six motions. The differences in range were attributed to location of the collar closure and initial head posture. The soft cervical collar showed that they restricted motion when worn either way.

It was disclosed in prior art that the rigid cervical collars were generally used to reduce cervical motion to a great extent than soft collars but the latter being preferred by the patients because of their greater comfort. In the article published in Spine (Phila Pa. 1976) in 2010 June 1; 35(13):1271-8. doi: 10.1097/BRS.0b013e3181c0ddad titled ‘Soft and rigid collars provide similar restriction in cervical range of motion during fifteen activities of daily living’, authored by Miller C P et al., it was stated that though there was data suggesting that the soft collars restricted full, active ROM (i.e., the extremes of motion), though to a lesser extent than the rigid braces. The article disclosed comparative studies assessing the effects of these two types of cervical collars on the functional ROM that is required to perform multiple activities of daily living. It was concluded that though the subjects exhibited a less full, active ROM of the cervical spine when a rigid collar was used, there was no significant difference in the motion in nearly all of the activities regardless of which cervical collar was applied. It was suggested that the small difference was due to both collars serving as proprioceptive guides that allow patients to regulate their own cervical motion based on their level of comfort.

The prior art, however, presents a few problems in using rigid or soft cervical collars for neck trauma. The hard cervical collars show highest degree of immobilization of the neck but can also cause adverse affects like tissue ischemia, exacerbation of pain, increased risk of aspiration etc. The soft cervical collars are flexible and accommodating to the body contours of the patient's neck, thus increasing the comfort levels but do not have the necessary rigidity in giving immobility to the neck.

The present invention is an attempt to address these issues and give a plausible and practical solution. The present invention attempts to combine the useful features of both the rigid and soft cervical collars into one device.

### OBJECTIVE OF THE INVENTION

The objective of the present invention is to provide a method of using a double soft collar that has a combination of desirable properties which being rigidity, similar to the hard cervical collar, and flexibility and softness, similar to the soft cervical collars.

Another objective of the present invention is to compare the usefulness of double soft cervical collar over the single soft and the hard cervical collars.

Yet another objective of the present invention is to show that the effectiveness of the double soft cervical collar is on par with that of the hard (Philadelphia collar) cervical collar.

### SUMMARY OF THE INVENTION

The present invention discloses a method of using a double soft collar and demonstrates its usefulness over hard (Philadelphia collar) and soft cervical collars.

One embodiment of the present invention relates to the method of using the double soft cervical collar comprising of wearing
i. a first single soft cervical collar
ii. a second soft cervical collar, on top of the first soft collar.

Another embodiment of the present invention relates to the method of using the double soft collar comprising of wearing
i. a first single soft cervical collar in a reverse manner with the sticky portion to the front,
ii. a second soft cervical collar, on top of the first soft collar, in a traditional manner with the sticky portion to the back of the neck.

Another embodiment of the present invention relates to the method of using the double soft collar comprising of wearing
i. a first single soft cervical collar in a traditional manner with the sticky portion to the back,
   ii. a second soft cervical collar, on top of the first soft collar, in a reverse manner with the sticky portion to the front of the neck.

Another embodiment of the present invention relates to the method of using the double soft collar comprising of wearing
i. a first single soft cervical collar in a traditional manner with the sticky portion to the back,
ii. a second soft cervical collar, on top of the first soft collar, in the reverse manner with the sticky portion to the front of the neck;
such that it creates a subjective feeling of tightness and inability to move the neck.

Another embodiment of the present invention relates to the method of using the double soft collar comprising of wearing
i. a first single soft cervical collar in a traditional manner with the sticky portion to the back,
ii a second single soft cervical collar, on top of the first soft collar, in the reverse manner with the sticky portion to the front of the neck;
such that it creates a subjective feeling of tightness and inability to move the neck; and that the feeling of tightness equal to the hard collar and the inability to move the neck is to the same extent of difficulty as that of the hard collar.

In yet another embodiment, the present invention relates to the usefulness of the double soft collar is in immobilizing the neck similar to the hard collar while being flexible like the single soft cervical collar.

In yet another embodiment, the present invention relates to the usefulness of the double soft collar is in being flexible and soft as the single soft cervical collar while immobilizing the neck like the hard cervical collar.

In yet another embodiment of the present invention, the double soft cervical collar presents less opportunity to cause adverse effects.

In yet another embodiment of the present invention, the double soft cervical collar reduces the possibility of adverse effects that are observed in the usage of the hard cervical collar like tissue ischemia, exacerbation of pain, increased risk of aspiration.

### BRIEF DESCRIPTION OF DRAWINGS

The following drawings have been provided along with the detailed description of the present invention by way of illustration only for a better comprehension of the procedure, and thus are not limitative of the present invention, wherein:

FIG. 1: shows a single soft collar worn in traditional manner with the sticky portion to the back.

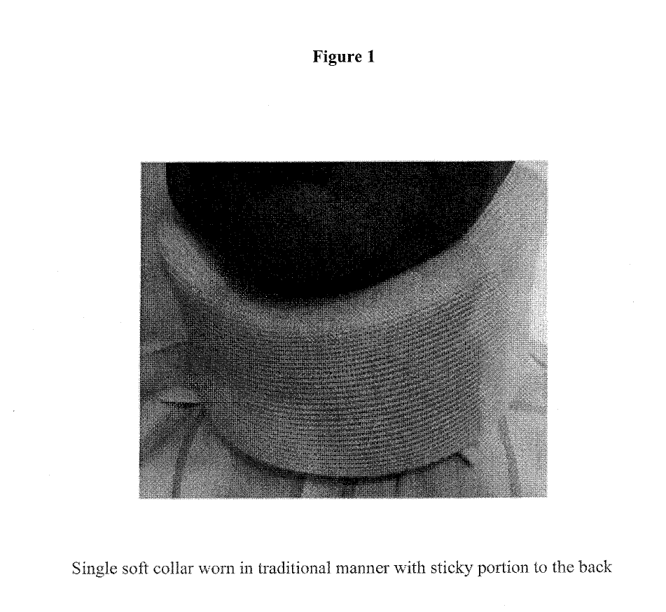

FIG. 2: shows single soft collar worn in the reverse manner with the sticky portion to the front.

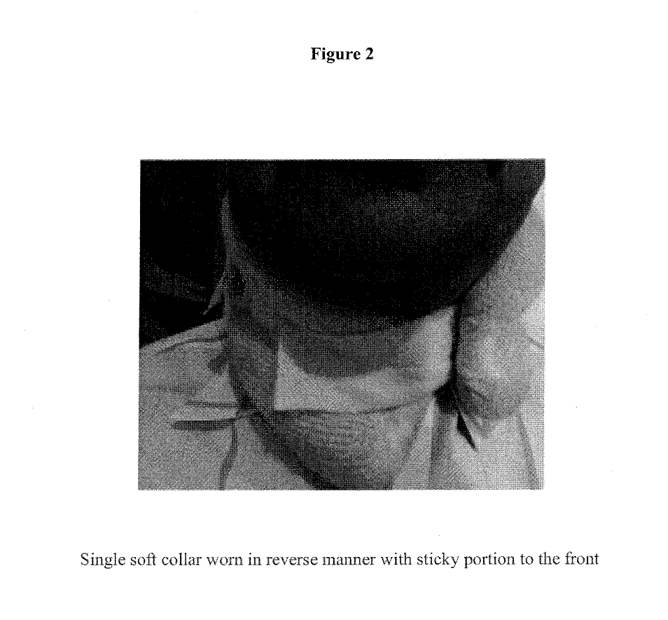

FIG. 3: shows a double soft collar with two soft collars worn in a traditional and reverse manner together.

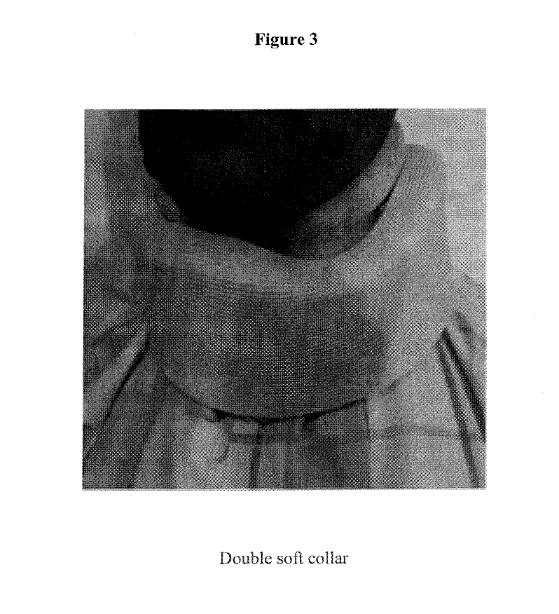

FIG. 4: show the graph depicting the results of the experimental study done for the tightness of different collars around neck.

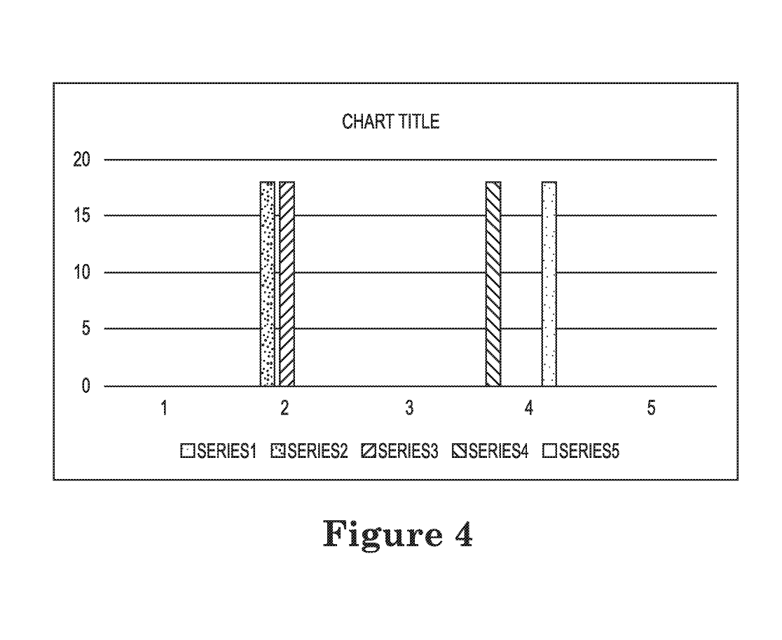

FIG. 5: shows the graph depicting the results of the experimental study done for the inability to move the neck with different collars around the neck.

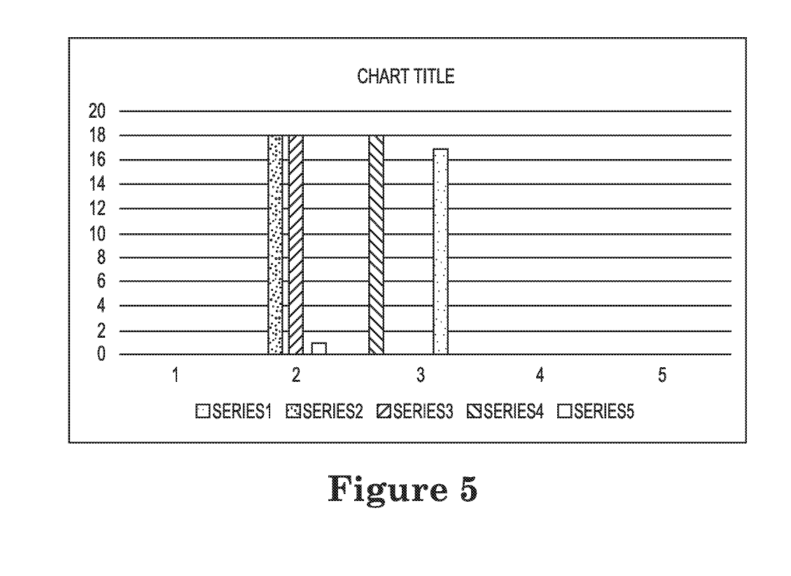

### DETAILED DESCRIPTION OF THE INVENTION

The present invention relates to the method of using a double soft collar in a manner that shows tightness around the neck and immobilizes the neck.

In one embodiment of the present invention, the method of using the double soft collar is disclosed, the said method comprising the following steps:
i. a first single soft cervical collar in a traditional manner with the sticky portion to the back,
ii. a second soft cervical collar, on top of the first soft collar, in the reverse manner with the sticky portion to the front of the neck.

In another embodiment of the present invention, the experimental method that shows the usefulness of the double soft collar is disclosed, the method comprising of the following steps:

A) Selection of Collars to be compared:
The collars selected for the present invention were of four types—hard cervical collars, single soft cervical collars worn in traditional manner, single soft cervical collars worn in reverse manner, and double soft cervical collars.

B) Selection of the Parameters to be tested for:
The parameters that are tested in the present invention were the subjective feelings of tightness around the neck and inability to move the neck.

C) Selection of Subjects:
The subjects selected for the experimental method of the present invention were the ER residents and attending physicians willing to participate in the experiment recruited on a voluntary basis. The exclusion criteria applied while selecting the subjects include excluding the ER residents and attending physicians unwilling to participate and the volunteers with any neck pain due to trauma or unspecified origin. In the present invention, 18 residents were recruited.

D) Preparation of Score Sheet:
Based on the parameters which were being tested, a simple tabulated recording sheet for the participants was designed for each individual participant which consisted of subjective feelings of tightness around the neck and inability to move the neck as the parameters and a scores ranging from 1 to 4 for each parameter which were meant to be circled.

E) Method of Experiment:
In the present invention, all the subjects selected were asked to wear the four collars one after the other. They were then asked to score their subjective feelings of tightness around the neck and inability to move the neck with regard to all the four cervical collars in the score sheets.

F) Recording the Results:
The results of the experiment of the present invention were recorded, tabulated and represented by means of graphs.

G) Description of scores:
The scores in the experiment for the present invention ranged from 1 to 4, for each parameter which were described as follows:
Tightness around the neck:
Score 1: No tightness
Score 2: Slightly tight
Score 3: Almost tight
Score 4: Very tight.
Movement of the neck upon wearing different cervical collars:
Score 1: Movement of the neck freely.
Score 2: Movement of the neck with slight difficulty.
Score 3: Movement of the neck with great difficulty
Score 4: No movement of the neck.

H) Tabulating Results for Tightness:
The results of the experiment conducted to compare the usefulness of the double soft collar in Watts of tightness around the neck, were recorded into Table 1 as follows:
a) For Hard Cervical Collar:
   Tightness around the neck was score 4 by all the 18 participants.
b) For Single Soft Cervical Collar worn in Traditional Manner:
   Tightness around the neck was score 2 by all the 18 participants.
c) For Single Soft Cervical Collar worn in Reverse Manner:
   Tightness around the neck was score 2 by all the 18 participants.
d) For Double Soft Cervical Collar:
   Tightness around the neck was score 4 by all the 18 participants.

TABLE 1

| GROUPS | SCORE 1 | SCORE 2 | SCORE 3 | SCORE 4 |
|---|---|---|---|---|
| A | 0 | 0 | 0 | 18 |
| B | 0 | 18 | 0 | 0 |
| C | 0 | 18 | 0 | 0 |
| D | 0 | 0 | 0 | 18 |

Groups - A: Hard collar; B: soft collar - sticky portion to the back; C: soft collar - sticky portion to the front; D: double cervical collar
Scores - 1: no tightness; 2: slightly tight; 3: almost tight; 4: very tight

I) Tabulating Results for Movement of Neck:
The results of the experiment conducted to compare the usefulness of the double soft collar in terms of movement of neck, were recorded into Table 2 as follows:
a) For Hard Cervical Collar:
   Movement of the neck was scored 3 by all the 18 participants.
b) For Single Soft Cervical Collar worn in Traditional Manner:
   Movement of the neck was scored 2 by all the 18 participants.
c) For Single Soft Cervical Collar worn in Reverse Manner:
   Movement of the neck was scored 2 by all the 18 participants.
d) For Double Soft Cervical Collar:
   Movement of the neck was scored 3 by 17 participants while 1 participant scored 2.

TABLE 2

| GROUPS | SCORE 1 | SCORE 2 | SCORE 3 | SCORE 4 |
|---|---|---|---|---|
| A | 0 | 0 | 18 | 0 |
| B | 0 | 18 | 0 | 0 |
| C | 0 | 18 | 0 | 0 |
| D | 0 | 1 | 17 | 0 |

Groups - A: Hard collar; B: soft collar - sticky portion to the back; C: soft collar - sticky portion to the front; D: double cervical collar
Scores - 1: move the neck freely; 2: move the neck with slight difficulty; 3: move the neck with great difficulty; 4: cannot move the neck at all

J) Analysis of the Results:
The values of the Chi-Square values and two tailed p-values after analysis were disclosed to be as follows:
a) Tightness around neck:
   Chi-Square value is 24
   Two tailed p-value is 0.0001.
b) Inability to move the neck:
   Chi-Square value is 58.7734
   Two tailed p-value is <0.0001.

K) Conclusions drawn form the Experiment:
a) It is disclosed that the double soft collar has the similar rigidity as shown by a hard cervical collar in regard to the inability to move the neck. It also discloses that the tightness of the double soft collar around the neck is also similar to the hard cervical collar.
b) The results of the experiment were compared and analysed for both tightness around the neck and movement of the neck after wearing the collar. The analysis was done using the Pearson Chi-Square analysis with two tailed p-value for both the subjective feelings.
c) It can be concluded from the experiment that the use of single soft cervical collar is less effective than the hard and double soft cervical collars. It was also concluded from the experiment that the double soft cervical collar is as effective as the hard cervical collar and that it can be used in traumatic conditions with additional reinforcements according to the need and the type of trauma involved.

1. A method of using a double soft collar and its usefulness over hard and soft cervical collars.

2. A method of using the double soft cervical collar, according to claim 1, comprising of wearing
i. a first single soft cervical collar
ii. a second soft cervical collar, on top of the first soft collar.

3. A method of using the double soft collar, according to claim 1, comprising of wearing
i. a first single soft cervical collar in a reverse manner with the sticky portion to the front,
ii. a second soft cervical collar, on top of the first soft collar, in a traditional manner with the sticky portion to the back of the neck.

4. A method of using the double soft collar, according to claim 1, comprising of wearing
i. a first single soft cervical collar in a traditional manner with the sticky portion to the back,
ii. a second soft cervical collar, on top of the first soft collar, in a reverse manner with the sticky portion to the front of the neck.

5. A method of using the double soft collar, according to claim 4,
ii. wherein the double soft collar creates a subjective feeling of tightness and inability to move the neck.

6. A method of using the double soft collar, according to claim 5,
wherein the feeling of tightness is equal to a hard collar and the inability to move the neck is to the same extent of difficulty as that of the hard collar.

7-12. (canceled)
